(19) United States
(12) Patent Application Publication
(10) Pub. No.: US 2002/0182585 A1
(43) Pub. Date: Dec. 5, 2002

(54) Combination and method using EDTA, cystine, zinc and selenium for anti-thrombin effect and for anti-platelet aggregation and measurement of efficacy

(76) Inventors: George Kindness, Middletown, OH (US); F. Timothy Guilford, Palo Alto, CA (US); Brooke Schumm III, Ellicott City, MD (US)

Correspondence Address: BROOKE SCHUMM, III, DANEKER, MCINTIRE, SCHUMM, PRINCE, GOLDSTEIN, ET A, 210 N CHARLES ST, SUITE 800, BALTIMORE, MD, 21201, US

(21) Appl. No.: 10/040,713

(22) Filed: Jan. 8, 2002

Related U.S. Application Data
(60) Provisional application No. 60/260,736, filed on Jan. 10, 2001.

(51) Int. Cl. C12Q 1/00; A61K 33/32; A01N 59/16; A61K 33/04; A01N 59/02; A61K 31/355

(52) U.S. Cl. 435/4; 424/702; 424/641; 514/458

(57) ABSTRACT

The invention is for the combination of EDTA, cystine, selenium, Vitamin C, Vitamin E, and zinc for anti-thrombotic effect and for the effect of restoring platelet aggregation, an integral component of thrombus formation, to normal and for the monitoring of the response to therapy with the combination. Methods for use of the components and method for performing the monitoring are included. The combination and method are particularly efficacious for vascular deficiency ailments including atherosclerotic vascular disease, reduction of ischemic cerebal event, complications from surgical procedures including restenosis, neurogenerative disease, and erectile disfunction, and vascular deficiency resulting from etiology of sepsis and chronic infection.

### CONTINUATION DATA

This is a continuation-in-part of Provisional Application 60/260,736 of this name filed Jan. 10, 2001, and a Provisional Application filed on Jan. 8, 2002, and priority is claimed from such applications.

### SUMMARY OF INVENTION

The invention proposes the use of with ethylene diamine tetraacetic acid (“EDTA”) and cystine, along with other compounds, and a method of use with a measurement of efficacy, for treatment of vascular deficiency diseases, or vascular deficiency for other diseases, sepsis or chronic infection.

### BACKGROUND

Vascular disease, in particular coronary artery disease (CAD) is the leading killer of men and women in the western world. Vascular problems, including those triggered by inflammatory processes, further contribute to an array of vertebrate afflictions. Interventions by vascular procedures, while potentially salutary in effect in the longer term, often yield inflammatory responses. Absent appropriate intervention with ethylene diamine tetraacetic acid (“EDTA”) in combination with other compounds and substances which will be discussed, platelets undergo a metamorphosis which leads to a “sticky” effect accompanied by activation of the intrinsic coagulation cascade. At the same time, absent appropriate measurement techniques, the end-point of therapy is not clear and patients are subjected to unnecessary treatment and cost, or, even worse, inappropriate treatment.

Significant benefits are available to patient populations through the use of EDTA chelation therapy and cystine which have not been formerly appreciated. The combination and method can accelerate the evolution of improvement in a patient population.

Prior art discusses the efficacy of MgEDTA and Na₂EDTA in conjunction with atherosclerosis. However, other literature references numerous patient disorders associated with vascular problems, but that has no reference to EDTA and cystine. There are a number of important areas in which this invention will have benefits:

Amelioration of atherosclerosis/vascular disease

Reduction of incidence of complication after vascular intervention such as angioplasty or other vascular surgery, including the slowing of restenosis

Reduction of incidence of stroke through anti-thrombin-platelet effect

Therapy for patients with neurogenerative disorders associated with decreased vascular supply such as Alzheimer's disease

Transient ischemic attacks, usually from decreased blood flow to the brain

Memory loss and inability to concentrate.

Another benefit, not necessarily confined to those with disorders traditionally associated with aging, is to permit improvement of the condition of erectile disfunction secondary to vascular insufficiency.

A different area of benefit is in the reduction of undesirable clotting resulting from sepsis or chronic infection.

The listed series of ailments to which these benefits relate will be collectively referred to as vascular insufficiency, including the ailments which are secondary to vascular insufficiency such as erectile disfunction, and including vascular insufficiency which is an effect of some other underlying etiology as in the case of sepsis.

While a recombinantly created analog of a naturally occurring human protein called protein C which has been given the trade name of Zovant, manufactured by Eli Lilly & Co., referenced in the “Wall Street Journal,” Jan. 4, 2001 issue page B2, could reduce undesirable clotting, the suggested price per dose is $5,000 to $10,000. EDTA and cystine treatment are several orders of magnitude cheaper per course.

Prior art, particularly, Rubin Martin, U.S. Pat. No. 5,114,974, May 19, 1992, suggests that EDTA can be efficacious in the amelioration of atherosclerosis. The proposed treatment in Rubin is to administer 3 grams of EDTA complex in a solution of 500 ml of 5% glucose (D₅W) for three hours. After administration, there would be three days rest. While the art suggests that zinc excretion in the urine will increase, no provision is made in the prior art to balance the associated body biochemistry with the increased zinc excretion as well as other essential trace elements, including Selenium, Manganese, Copper, Chromium and macronutrients to include Magnesium. The biochemical mechanism has been poorly understand and therefore no guidance is given as to how long treatment regimen should be continued nor are other components of biochemical imbalance addressed such as by this invention. For instance, the use of Na₂EDTA or MgEDTA causes depletion of zinc resources in the body, but the use of Zn⁺EDTA is not practical because the EDTA binding coefficent of Zn is higher than that of Na or Mg. See Table 1 showing the relative binding coefficients and illustrating the preferability of Na and Mg. The table is apparently adapted from Schwartzenbach, 1957. Scientific basis of EDTA chelation therapy, by Bruce Halsted, MD. Golden Quill Publishers, Inc. Box 1278, Colton, Calif. 92324.

TABLE 1

| | Stability | |
|---|---|---|
| | Constant | |
| Metal Cation | Log K | |
| Fe⁺⁺⁺ | 25.1 | Most Stable |
| Hg⁺⁺ | 21.8 | |
| Cu⁺⁺ | 18.8 | |
| Pb⁺⁺ | 18.5 | |
| Ni⁺⁺ | 18.0 | |
| Zn⁺⁺ | 16.5 | |
| Cd⁺⁺ | 16.5 | |
| Co⁺⁺ | 16.3 | |
| Al⁺⁺ | 16.1 | |
| Fe⁺⁺ | 14.3 | |
| Mn⁺⁺ | 13.7 | |
| Ca⁺⁺ | 10.7 | |
| Mg⁺⁺ | 8.7 | Least Stable |

Present therapy after vascular intervention procedures often involves the use of “blood thinners” like coumadin and aspirin, none of which are healthful as a long-term proposition, having various side effects which effects are often more pronounced in the very population most in need of the proposed therapy in this invention. The “blood thinners” have a significant side effect of permitting “blood leakage” in the brain, often leading to strokes. Non-steroidal anti-inflammatory drugs (NSAID's), including aspirin, do not have an effect on thrombin induced clotting of blood platelets.

Present technology does not easily provide a system in conjunction with use of EDTA for objective measurement of adequate improvement in platelet clumping (aggregation) characteristics of a patient. The present art only enables indirect measurement of success by the use of an angiogram to inspect the openness and diameter of the artery, an expensive and risky procedure.

The invention has the following general objects. Addressing these general objects will yield the benefits in addressing the disorders just mentioned. Those general objects are:

1) Minimization of inflammatory response after vascular incident or vascular intervention procedures

2) Improvement of immune system competency

3) Measurement of appropriate endpoints for discontinuing therapy

4) Maintenance of proper body biochemistry, physiological function, including eliminating redox imbalance, and restoration of mineral balancing.

5) By increasing available glutathione, prevention of depletion of glutathione which depletion increases the propensity of “foam cells” to form and participate in arterial plaque formation.

6) Reduction of plasma calcium with respect to plaque formation

The key anti-oxidant contemplated which has a variety of positive biological effects is cystine or NAC or other glutathione pathway enhancing and detoxifying compounds later described. The avoidance of a glutathione deficiency steers the patient to have a higher Th-1 response to Th-2 response ration that the patient would have with any glutathione deficiency. Peterson, J. et al, “Glutathione levels in antigen-presenting cells modulate Thl versus Th2 response patterns,” Vol 95(6), Proceedings Nat'l Acad. Sci. USA p. 3071-76 (Mar. 17, 1998).

The background chemistry relates first to overall control mechanisms that rely on the regulation of “Free Radical Mechanisms” and the production of reactive oxygen species (ROS) and reactive oxygen intermediaries. Sources of ROS include heavy metals, pesticides, drugs, diet; activated leukocytes, enzymes, xenobiotics from indoor and outdoor air, for example—cigarette smoke, radon, O₃, NO₂, SO₂, car exhaust, x-rays, and ultraviolet, to name a few.

The shift of the oxidant/antioxidant balance, through free radical generation, in favor of oxidants in cells is termed oxidative stress.

The term antioxidant is frequently used in medical literature, and should be correctly defined as “Any substance that, when present in low concentration compared to that of an oxidizable substrate, significantly delays or inhibits oxidation of that substrate.

This definition is particularly relevant to the use of EDTA and in particular its therapeutic window in association with supplemental antioxidant/antiplatelet therapy. Thus antioxidants can act at different levels in an oxidation sequence to prevent, intercept or to repair (reverse) cell and tissue free radical injury.

Examples of Intracellular Antioxidants are as follows:

Superoxide Dismutases

Catalase

Glutathione Peroxidase

Glutathione

Examples of extracellular antioxidants are as follows:

Vitamin E & Selenium

EDTA

Vitamin C

Uric Acid

Cholesterol

Albumin

Sulphydryls

β-Carotene

A free radical is a molecule or molecular fragment that contains one or more unpaired electrons in its outer orbital. Radicals are formed by accepting or losing an electron or by homolytic fission of a covalent bond. In simple terms electrons in atoms occupy regions of space known as orbitals. Each orbital can hold a maximum of two electrons. A free radical is simply defined as any species capable of independent existence that contains one or more unpaired electrons occupying an orbital.

Molecular oxygen is a bi-radical, possessing two unpaired electrons in its outer orbital. In actively respiring cells, more than 90% of molecular oxygen is completely reduced by mitochondrial cytochrome oxidase in a four electron (Tetravalent pathway) with water as the end product.

The univalent and sequential reduction of O₂ (univalent pathway), results in the formation of oxygen-derived free radicals.

The first reduction of O₂ results in the formation of the superoxide anion radical. The subsequent reduction product is hydrogen peroxide (H₂O₂) which is a reactive oxygen intermediate (ROI) but does not have the structure of a radical.

Another reduction product of O₂ is the hydroxyl radical which may result from the interaction of O₂− with H₂O₂ in the presence of iron (Fenton reaction). The hydroxyl radical is highly toxic and reacts immediately with most biological systems.

The last reduction of O₂ results in the formation of water. To summarize:

O2−+e→O2.− Superoxide anion

O2HO→HO.+OH− Hydroperoxyl radical

HO+e−+H+→H2O2 Hydrogen peroxide

H2O2+e−→.OH+OH− Hydroxyl radical

Reactive oxygen species (ROS) are important mediators of cell and tissue injury (see figs.), and are the major players in the process of aging and apoptosis.

Thus oxygen-derived free radicals—superoxide anion, (O2.−), hydroxyl radicals OH. or metabolites such as hydrogen peroxide and hypochlorous acid (HOC1) must be regulated.

Activation of neutrophils is a natural part of the body defense mechanism. The activation generates O2− which is rapidly converted to H₂O₂ by superoxide dismutase (SOD). However, it must be remembered that OH. formed non-enzymatically in the presence of Fe₂+ or superoxide anion reacts with iron and copper to form hydroxyl radicals, Note: This is an important role for EDTA and other chelators as regulators of Fe/Cu generated free radicals. Also in neutrophils, myeloperoxidase results in the formation of HOC1 from H₂O₂ in the presence of chloride ions.

Another free radical is produced during normal physiological processes, this is nitric oxide (NO.). Nitric oxide is produced by the vascular endothelium and RELAXED vascular smooth muscle. It is also produced by phagocytes and epithelial lung cells whereby

the reaction may protect lung cells against (O2.). However, O2.−+NO.→ONOO.− (PEROXYNITRITE), which is a strong oxidant and contributes to lung injury.

Excess (ONOO.−) may be produced when cytokines have increased production of both (NO.) and (O2.−). At physiological pH peroxynitrate causes direct damage to proteins, and decomposes into toxic products that include nitrogen dioxide and hydroxyl radicals.

The multiple effects of nitric oxide particularly with respect to lung function emphasizes the role of free radicals in homeostasis, inflammation and oxidative stress.

In summary:

| | Oxygen species |
|---|---|
| Primary Reaction | |
| Superoxide Anion | (02•-) |
| Hydroxyl Radicals | (OH•) |
| Hydrogen Peroxide | (H2O2) |
| Singlet Oxygen | (O) |
| Nitric Oxide | (NO•) |
| Peroxynitrite | (ONOO•-) |
| Secondary Reactive | |
| Peroxyl Radical | (ROO•) |
| Alkoxyl Radical | (RO•) |

The benefits of anti-oxidant reactions can be summarized in the table below:

### Antioxidant Actions

1. Prevent the formation of free radicals or initiation of peroxidation by scavenging free radicals.

2. Conversion of oxidant to less toxic free radicals.

3. Compartmentalization of reactive oxygen species away from vital cellular structures.

4. Repair of molecular injury induced by free radicals.

5. Binding of metal ions in forms that will not generate reactive species.

6. Removal of peroxides by conversion into non radical products such as alcohol.

7. Breaking chain reactions, i.e. reacting with chain propagating radicals (peroxyl and alkoxyl).

It is important to realize that by age thirteen years almost everyone has fatty streaks in their vessels. The fatty streaks can progress to atherosclerotic plaque that start out as accumulations of lipid in the form of oxidized low-density lipoprotein (LDL) within macrophages or foam cells, and other monocytes. Chronic minimal injury to the arterial endothelium is physiological and results from disturbances in the pattern of blood flow in parts of the arterial tree, such as bending points and areas near bifurcations. Monocyte inflammatory cells are recruited by the release of local cell stress factors such as intracellular adhesion molecule (ICAM-1). Additional factors associated with chronic injury include hypercholesterolemia, diabetic related biochemical alteration, chemical irritant such as tobacco smoke, hypertension related biochemicals such as vasoactive amines, immune complexes and infections.

Most lipids deposited in atherosclerotic lesions are derived from plasma low-density lipoprotein (LDL) that enter the injured or dysfunctional vessel lining, the endothelium. LDL is subsequently oxidized and modified. The oxidized LDL combines with other factors to increase the inflammatory response. High density lipoprotein (HDL) plays a protective role in the LDL oxidation and accumulation.

The combination of these events results in platelet activation and aggregation, which in turn leads to clot formation (thrombus). Lipid rich plaques are often vulnerable to disruption, which in turn leads to further progression of the formation of thrombus, with an increased platelet deposition. As a vessel narrows the narrowed space creates an increase in the rate of blood flow and an increase in shear stress on the platelets, furthering the formation of clot.

Dietary alterations aimed at lowering plasma cholesterol and LDL levels presumably modify the lipid rich plaque and have been shown to lower the incidence of acute myocardial events. However, only minimal regression of atherosclerosis has been shown with dietary modification (1% to 2% decreases in the degree of stenosis).

Therefore, Free Radical Pathology is the major instigator of vascular disease. The primary intracellular defenses to deal with free radical pathology and oxidative stress are catalase, superoxide dismutases, and the enzymes of the glutathione redox system. The intracellular actions are supplemented through the use of chelation therapy and appropriate antioxidant supplementation.

Antithrombotic and anticoagulant agents have been observed to be beneficial in the prevention of acute coronary events. While aspirin is the most widely used antithrombotic agent, aspirin interferes with only one of the pathways of platelet aggregation (thromboxane A2).

The aggregating stimuli unaffected or least modified by aspirin and/or associated NSAID:

1. ADP (ADENOSINE—5′-DIPHOSPHATE) and Collagen dependent aggregation pathway

2. Thrombin dependent pathway

3. Coagulation cascade

Current anticoagulation agents interfere only partially with the coagulation system and do not affect platelet aggregation. It is no surprise, therefore, that aspirin, plavix and anticoagulants cannot completely prevent coronary thrombotic events due to their limited mechanism of action.

High-risk patients are currently being advised to consider combination therapy with a Platelet inhibitor (aspirin or ticlopidine) and an anticoagulation agent such as heparin or coumadin. Only short-term therapy (1-3 weeks) with combination anticoagulants is recommended at this time.

Intravenous materials are under study to block thrombin related platelet aggregation, but so far no especially efficacious combination has been disclosed.

Important to the understanding of thrombin related platelet aggregation is the reaction of the blood platelet. The reaction of blood platelets is the single most important event in vascular disease. When the platelet undergoes insult or injury this causes a release reaction to take place resulting in the platelet undergoing both a change of shape and becoming sticky. In areas of reduced blood flow these cell fragments come together to form aggregates. The small aggregates migrate to larger vessels whereby they interact with plaque to form a blockage. Any inflammation aggravates the propensity to blockage. Several agents which are noticeably involved in this process can be readily monitored and the effects of EDTA on this process as well as the series of events involved in CAD/Atherosclerosis documented. Thus, by examining the following agents the series of events leading to cardiovascular events can be assessed:

Adenosine 5′ Diphosphate (ADP):

This substance is contained within storage granules within the platelet. Under stress, or in the presence of appropriate stimuli, ADP can be released from the platelet. This triggers the platelet to undergo the process of viscous metamorphosis (shape change-sticky) that results in more platelets adhering to each other. This process is reversible and is inhibited by EDTA as well as aspirin and other non-steroidal anti-inflammatory drugs (NSAID's). Thus, if an individual is self medicating either through the use of NSAID or Plavix by prescription, then ADP-induced platelet aggregation provides a monitor of therapy. Similarly the influence of various medications and their possible interaction with EDTA can effectively be overseen.

Epinephrine:

Epinephrine, like ADP, is contained within the storage granules. Under condition of stress, epinephrine release with the concomitant release of ADP will trigger the activation of clotting. Additionally, epinephrine from within the storage granules will contribute to the vasoconstrictive effects of catecholamines. As with ADP, the monitoring of epinephrine allows for the evaluation of aspirin NSAID effects. Vitamin B-complex and EDTA have a noted “calming” effect on these platelet responses.

Collagen:

The response of the blood platelet to the vessel wall is dependent on collagen. The collagen response is independent of calcium. However, if there is a blockage of the collagen receptor, the collagen-platelet effect will be inhibited. This phenomenon is also observed when the cell membrane becomes damaged as would occur in free radical pathology or with lipid peroxidation. The collagen response, independent of calcium, reflects membrane receptor integrity. Similarly, a good collagen platelet aggregation response means that in the event of trauma the platelets can form a hemeostatic plug. The collagen-platelet response also reflects the antioxidant efficacy of ascorbic acid, because Vitamin C is required for effective collagen cross-linking.

Thrombin:

The most important anti-thrombotic and anti-platelet effect of EDTA is seen in the inhibition of thrombin induced platelet aggregation. This test as such attests to the efficacy of EDTA in the treatment of vascular disease. How this is done will be addressed momentarily.

Preferred Mode of Invention:

The preferred mode of the invention proposes the use of one of either Na₂EDTA or MgEDTA in combination with a glutathione cycle enhancing compound, preferably cystine. The preferred mode is preferably used in combination with intermittent oral zinc and selenium therapy. Cystine is preferable to the alternative of Cysteine, or NAC, because it is more rapidly uploaded into the glutathione cycle and is thus more effective in preventing inflammatory response at the critical time, particularly in an invasive vascular intervention such as angioplasty. The surprising, though logical, effect that is yielded by the combination is that the reduction in inflammatory response, and/or increase in immune system competency, increases the effectiveness of the chelated EDTA and enables better patient recovery. This is further enhanced by the decreased likelihood of glutathione depleted foam cells. That recovery can be objectively ascertained by measurement of the glutathione level and by performing a platelet aggregation test. Those tests can be performed immediately prior to treatment. If performed after administration of a dose, then benefit will be seen, but the best means of measuring efficacy of the treatment is 12 to measure glutathione level and platelet aggregation immediately before commencement of administration of the 1 next dose. If improvement is noted at that time, and prior to commencement of subsequent doses, then a positive 1 trend can be ascertained. The suggested dose for an average adult is 1.5 to 3 g in 500 cc administered over 3 hours and 2-6 g of L-cystine with that dose. On the next day after the EDTA/cystine therapy, a zinc supplement of 25 mg-50 mg should be administered orally.

Dosing in patients with decreased kidney function is calculated using the Cockcroft-Gault Equation,

$$Creatinine Clearance ( CrCl ) = \frac{( 140 - Age ) \times Wt in Kg . )}{( 72 \times serum Creatnine )}$$

EDTA dose administered at each infusion=50 mg EDTA per (Weight in Kg)×(CrCl/100), up to a maximum of 3.0 g EDTA.

Abbreviations:

CrCl=computed renal glomerular filtration rate in ml/min

Age=patient's age

Cr=serum creatinine in mg/dL

For women, multiply the above result by 0.85

Oral administration of EDTA has been generally over looked as the absorption of EDTA is low. The use of oral EDTA may of use in maintenance and prevention even if the absorption is low. Oral dose suggested is 750 mg/day in a single dose administered at least one hour after the last meal of the day. Administration one hour after the last meal of the day prevents EDTA from binding essential minerals from foods and minimizes the likelihood of nutrient deficiency. EDTA may also be administered as a suppository at 500 mg. per day, with bedtime administration as the preferred dosing time. The term pharmaceutically acceptable carrier includes aerosol, intravenous, oral and rectal devices and other acceptable routes of administration through which EDTA, and other compounds in this invention, such as cystine, zinc, selenium, vitamin E and vitamin C can be administered, and excipients with said compounds.

The addition of cystine, cysteine, N-acyl cysteine, or the pharmaceutically acceptable salt of those substances yields another effect in this invention not facially evident from the independent properties of the basic components of the invention (hereafter each substance or a pharmaceutically acceptable salt is referred to as a “cystine family member”). The glutathione cycle is a critical body cycle whose importance has not been fully appreciated. Administration of a cystine family member, preferably cystine, which has the best and most rapid upload into the glutathione pathway, or N-acetyl-cysteine, enhances the immune system competency of the patient.

Cystine is (3,3′-dithiobis [2-aminopropanoic acid]). Cystine is readily reduced to cysteine. Cystine is present in most mammalian hair and keratin.

Cysteine is 2-amino-3-mercapto propanoic acid. It is readily converted by oxidation to cystine. It is a constituent of glutathione and abundantly present in the metallothionines.

Cystine in the body-useful form as L-cystine is available from Spectrum Chemical Mfg. Corp. 14422 S. San Pedro St., Gardena, Calif. 90248.

Cystine, cysteine, and N-Acetyl cysteine and pharmaceutically acceptable salts, including the pharmaceutically active forms described in Kozhemykin et al, published by WIPO as WO 00/031120, PCT/RU99/00453, filed internationally on Nov. 19, 1999, “Hexapeptide with the Stabilized Disulfide Bond and Derivatives Thereof Regulating Metabolism, Proliferation, Differentiation and Apoptosis,” will all collectively be referred to as cystine in this invention. Included in the term cystine is also any therapeutically beneficial sulfur donating compound, including ebselen, which interacts with the glutathione pathway. The invention contemplates in the term cystine undenatured whey protein products designed to have enhanced cystine concentration as well as protein products which contain cysteine and cystine. They can be in the form of food products.

Because of the ready biochemical conversion of cystine into glutathione the use of the term cystine will also refer to the use of glutathione directly in the intravenous form. Oral glutathione or s-acetyl-glutathione may also be used. As oral gluathione may be subject to digestion in the GI tract, the intravenous form is recommended. Intravenous glutathione is prepared in a similar fashion using reduced L-glutathione obtained from Spectrum Chemical Mfg. Corp. 14422 S. San Pedro St., Gardena, Calif. 90248. The process is performed using a layer of nitrogen gas to expel the excess oxygen overlying the liquid. This step is done to maintain an oxygen free environment that will limit the amount of oxidation that may occur during storage. The glutathione is diluted in sterile water or saline using 200 mg glutathione per ml of diluent. Administration of 200 mg. to 1200 mg per day is well tolerated.

Additionally, selenium should be administered orally on the next day after treatment in the amount of 200 μg to maintain adequate levels. Selenium is an important catalyst for glutathione peroxidase activity in the glutathione cycle enabling the capture and excretion of free radicals, especially hydroxyl radicals.

Administration of Vitamin C (1-5 g) and oral Vitamin E (800-1200IU) to maintain normal levels is also appropriate and to prevent any deficiency in those vitamins from interfering with the efficacy of the treatment protocol.

For a procedure such as angioplasty or other invasive vascular surgery, the reduction of inflammation and inhibition of thrombotic effect and platelet aggregation effect will accelerate the evolution of improvement in the patient's condition, and defer the onset of symptoms even in disorders not requiring or unable to be treated by invasive vascular procedures. In the instance of sepsis, the reduction of inflammation and inhibition of thrombotic effect and platelet aggregation effect will accelerate the evolution of improvement in the patient's condition, and defer the onset of destabilizing symptoms.

In the best mode, there should be measurement of glutathione level and a platelet aggregation test, as well as prothrombin time, activated partial thromboplastin time, total serum calcium, ionized calcium, total magnesium, ionized magnesium, β2-microglobulin and serum creatinine or creatinine clearance. The former, the glutathione level, is very difficult at most laboratories. The latter, the measurement of platelet aggregation, is not available at most laboratories. All other tests are routinely available in clinical laboratories. Measurement of glutathione can be done through a difficult process according to Tietze.

Method of Monitoring the Use of the Invention in the Treatment of Increased Platelet Agglutination and Vascular Disease:

The invention proposes semi-weekly monitoring of glutathione and platelet aggregation as treatment commences for two weeks, and then, assuming a non-negative trend in glutathione level and platelet aggregation, weekly monitoring for three months, and thereafter bi-weekly monitoring. Creatinine should be monitored initially and then every tenth administration of EDTA. The inventors also propose pre-treatment with cystine prior to any invasive vascular intervention procedure.

The purpose of the invention is to ultimately restore platelet aggregation characteristics to normal levels as set forth in the attached Table I entitled Coagulation Profile. A user should be mindful that creatinine clearance should be monitored to confirm proper kidney function. Because each patient can have unique characteristics and profiles, a baseline is suggested of a complete blood count, the preferred indicia of which are in the attached table II. A baseline chemistry profile including a Comprehensive Metabolic Profile, is also suggested per the attached Table III, and a baseline Lipid/Cardiac Risk is also suggested per the attached Table IV. Table V has a baseline chart to use for determining efficacy of kidney function for purposes of the invention. Table VI has a post-treatment chart of preferred biochemical results. Abbreviations should be known to those skilled in the art. Table VII is added in order to assist with abbreviations.

The inventors also recommend the monitoring of HDL cholesterol and LDL cholesterol and ferritin. See, Table VIIIA and VIII B. Mitigation of abnormal levels closer to normal ranges enables further evaluation of a patient's progress, though not as precisely predictive as the platelet aggregation indication tests set out in this invention.

Glutathione Level Test:

Determination of glutathione levels for plasma and/or red blood cells is the preferred test. The test is performed according to Tietze, 1968 Enzymic Method for the Quantitative Determination of Nanogram Amounts of Total and Oxidized Glutathione Analytical Biochemistry with an additional reference of Tietze, 2^{nd} ed., Chemical Chemistry 1994, pp. 1779-1780. This Tietze method has been modified as follows:

$$GSSG + DTNB + {H}^{+} {\to -> }^{GR } 2 G - SH + {DTN}^{+} \qquad ( 1 )$$

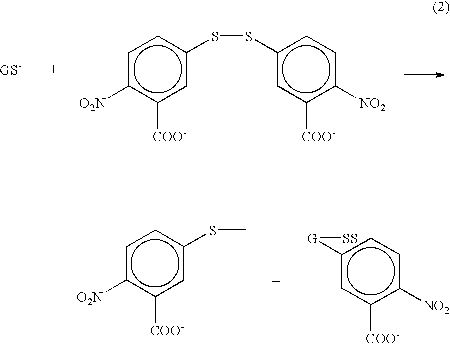

where GSSG is glutathione, oxidized

GR is glutathione reductase

DTNB is a sulfhydryl reagent 5,5′-dithiobis-(2-nitrobenzoic acid)

G-SH is glutathione, reduced

DTN⁺ is dithiobisnitrobenzoic acid

GS⁻ is a transition state between glutathione reduced and oxidized

The method of glutathione assay provides a sensitive method for total and oxidized glutathione. The modification increases sensitivity for spectrophotometric analysis. The reagents in use throughout this invention, including for this test, are either generally available from a chemical supply house or available from Sigma Chemical Co., Inc. or a company associated with it, Aldrich Chemical Company, of St. Louis, Mo. Incorporating DTNB, a sulfhydryl reagent 5,5′-dithiobis-(2-nitrobenzoic acid) in the first reaction which possesses a molar absorption at 412 mμ then forms two moles of GSH per mole of reduced nucleotide utilized in the GSSG reduction in reaction (2). The rate of chromophore development depends on the concentration of glutathione in the reaction mixture detectable to 10 nanograms ml⁻¹. This provides a highly sensitive and specific procedure for measuring glutathione. The normal level should be approximately 200-400 micromoles/liter for plasma and red blood cells. The test may be performed on an automated clinical chemistry analyzer (also called a random access analyzer) such as Roche Cobas Fara. Samples are collected carefully to prevent contamination. Frozen plasma collected from ACD, EDTA, and heparin may be used. The invention could test reduced glutathione but there is not any efficacy over testing total glutathione. Another means of testing glutathione is specifically referenced in Ellerby, L. et al, Measurement of Cellular Oxidation, Reactive Oxygen Species, and Antioxidant Enzymes During Apoptosis, 322 Methods in Enzymology 419-420 (Academic Press 2000).

A discussion of the therapeutic value of appropriate levels in the glutathione pathway is discussed in Rahman I, MacNee W, Free Radical Biological Medicine 2000, May 1, 28(9): 1405-1420.

Testing For Thrombic Propensity and Propensity to Platelet Aggregation:

A blood sample is taken and stabilized to prevent natural clotting. The blood is centrifuged. It is spun relatively slowly at approximately 1000 rpm for 15 minutes (360g (g=gravitational constant) so red blood cells (“RBC”) are at bottom leaving platelets suspended in plasma just above RBC's. The platelets are then pipetted off trying to have no RBC's in the pipette. The platelet rich plasma is then ready for testing. The pipetted platelets are tested with five different reagents in a cuvette comparable in size to a major artery. The reagents are:

1)ADP

2) Epinephrine

3) Collagen

4)Thrombin

5) the patient's blood with saline as a control

The cuvettes containing the platelets and selected reagent are run in a platelet aggregometer supplied by Helena Laboratories of Beaumont Texas referred to as a Kyoto Daiichi Kogaku Co. Ltd. (“KDK”) model Monitor IV Aggregation Recorder for 5 minutes. A magnet is placed in each tube before starting the timer to rotate the mixture in the cuvette and provide turbulence to imitate normal turbulence of blood flow.

After five minutes a printout is generated reflecting the five cuvettes. The graph runs from 0-5 minutes. The purpose of the graph is to show the relative aggregation during the five minute run. The reading is from 0-100% based on the clarity of the solution with 0 being the most turbid due to free platelets. Light in the aggregometer is less scattered if platelet aggregation occurs. The purpose of the visual inspection is to insure that if a large thrombus is blocking light, the test result is competent. A further purpose is to support a clinical opinion. The laboratory makes a visual observation of the separated platelet sample run with the reagent thrombin, selecting from the following list the best description, and when appropriate microscopic examination of the test cuvette contents.

1) Fibrin strand

2) Small single thrombus

3) Large single thrombus

4) Small fibrin clot

5) Large fibrin clot

6) Free floating single thrombus, with free platelets observed

7) Free floating fibrin strand, with free platelets observed

8) Single balloon thrombus with no free platelets

9) Free platelets-no clotting

Scoring System:

1-5 with 1 being the best response to respective challenge and 5 representing poor response to respective challenge.

The most commonly observed states are the following three: free platelets-no clotting, single thrombus, or large fibrin clot.

The printout is examined and interpreted to produce a score of one to five for the platelet sample, with one being the best score for patient health, and five the worst. One is the best rating and normally evidenced by positive graph results and free platelets-no clotting. The graphs, and a review of the primary diagnosis for the patient's sample, and known patient medication or graphical observations, enable the score to be adjusted for drug interactions such as a patient taking aspirin which may influence test results.

The graph produces a line that begins at a low point at the commencement of the time and may then be straight or normally curved upward, or some combination of the two. A general guide for reading the results is as follows: If the test is done on a pre-treatment sample, and the graph line for thrombin is straight, and there is a fibrin clot or a large thrombus, then the patient is rated a five.

The most desirable result, and one showing successful treatment, is a straight line for the ADP, epinephrine and thrombin reagents, and observed free platelets. Such a result is rated a one. This implies inhibition of aggregation.

If the line is straight with respect to collagen, then the sample is usually rated a 4 due to interference with the collagen receptor. If there is a fibrin clot or a large thrombus, and a straight line for the thrombin reagent, then the patient usually receives a score of 4 or 5. If the graph for thrombin shows a biphasic curve, then the result is average and usually graded a three. In that instance, the observation of the cuvette is usually that fine to medium aggregates are seen in the samples run with reagents ADP, epinephrine or collagen. In the cuvette having the sample run with thrombin, a single thrombus is normally seen in that instance. In certain instances, there is a straight line in the sample run with thrombin that then rises and forms a curve. That shows a delay >60 sec. in inhibition of aggregation with a biphasic response and usually rates a score of 2-3. This is often seen in a post-treatment sample after a treatment but prior to completion of therapy.

In general, if the thrombin graph line is not straight and there is no visual observation with clot-free platelets, there is usually a fibrin clot or a single large thrombus suggesting a rating of four to five and the patient should continue the therapy described in this invention.

For a separated platelet sample in a cuvette with the collagen reagent, if there is a straight line, and other data or the tube observations suggest a propensity to aggregation, those results suggest that a drug interaction such as acetaminophen, e.g. Tylenol (registered trademark of Johnson & Johnson) or aspirin may be present. If the line for ADP rises and then proceeds in a straight line, this indicates the patient is under oxidative stress, and the patient is scored as a 4 unless other data shows a more negative score is appropriate.

If a rise of curve is relatively smaller for and then straight for the ADP or epinephrine reagents, the sample usually is scored a 3. A yet smaller rise could be scored as a 2.

If there is any visual observation aside from free platelets, or a single large thrombus, and, for instance the graph as to the thrombin reagent proceeds 20-40-60-80 this implies a delay in the onset of thrombin induced aggregation.

Ideally, lines that show no thrombus or fibrin clot in the thrombin reagent cuvette, and a lack of aggregation, meaning a reading of zero, and where there is visual observation of free platelets, the patient should have a favorable rating of one. Straight lines that show only free platelets in ADP and epinephrine also rate a most favorable patient health rating and show inhibition of epinephrine-induced or ADP-induced platelet aggregation.

The control will show that the mechanical components of the machine are working properly because there should be no alteration in platelet function by the addition of a small amount of saline. No alteration in platelet aggregation should be apparent in that cuvette.

Saline additionally acts as a patient control whereby if the test platelets are subject to spontaneous aggregation, e.g. “stress type” reactions, this will be evidenced by the formation of aggregated platelet clumps. This response being abnormal reflects an increased propensity for clot formation.

This description and the directions for the KDK Monitor IV Aggregation Recorder, which are incorporated by reference, should enable a reasonably skilled person to determine the success of treatment and the success during the course of treatment by measuring platelet aggregation.

The term “therapeutic dose” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. A consideration of these factors is well within the purview of the ordinarily skilled clinician for the purpose of determining the therapeutically effective or prophylactically effective amount.

The concept and invention is not meant to be limited to the disclosures, including best mode of invention herein, and contemplates all equivalents and permutations to the invention and similar embodiments to the invention for humans and mammals and veterinary science. Equivalents include all pharmacologically active racemic mixtures, diastereomers and enantiomers of the listed compounds and their pharmacologically acceptable salts.

TABLE I
BASELINE-COAGULATION PROFILE

| TEST | NORMAL RANGE | UNITS |
|---|---|---|
| Prothrombin Time PT | 11-15 | sec. |
| Activated Partial | 16-25 | sec. |
| Thromboplastin time (APPT) | | |
| Fibrinogen | 200-400 | mg/dL |

Platelet Aggregathon Score:

| | | Normal score |
|---|---|---|
| | | under invention |
| Reagent | | criteria |
| Adenosine 5'diphosphate | ADP | 3 or less |
| Epinephrine | EPI | 3 or less |
| Collagen | Coll | 3 or less |
| Thrombin | THR | 3 or less |

TABLE II
BASELINE-CBC/Complete Blood Count

| TEST | NORMAL RANGE | | UNITS |
|---|---|---|---|
| WBC | 5-10 | | Thou/CMM |
| RBC | M: 4.6-6.2 | F: 4.2-5.4 | M/μL |
| HgB | M: 14-18 | F: 12-16 | g/dL |
| HCT | M: 40-54 | F: 37-47 | % |

| | | |
|---|---|---|
| MCV | 82-99 | FL |
| MCHC | 33-36 | g/dL |
| RDW | 11.5-14.5 | |
| PLT | 150-400 | k/μL |
| MPV | 6.2-10.8 | FL |
| Lymph | 25-40 | % |
| Mono | 1-8 | % |
| Baso | 0.5-1.0 | % |
| Eosin | 1-4 | % |
| Segs | 50-70 | % |

TABLE III
BASELINE-SMAC/Metabolic Comprehensive Profile

| TEST | NORMAL RANGE | UNITS |
|---|---|---|
| Alkaline Phosphatase | 30-103 | U/L |
| Bun | 7-19 | mg/dL |
| Creatinine | 0.7-1.4 | mg/dL |
| Bun/Creatinine | | |
| Glucose | 64-112 | mg/dL |
| Total Protein | 6.0-8.2 | g/dL |
| Uric Acid | 2.1-6.1 | mg/dL |
| Albumin | 3.8-5.2 | g/dL |
| Albumin/Globulin Ratio | | |
| Calcium | 8.5-10.1 | mg/dL |
| Phosphorus | 2.4-4.2 | mg/dL |
| Sodium | 136-145 | mmol/L |
| Potassium | 3.5-5.1 | mmol/L |
| Chloride | 95-106 | mmol/L |
| Bilirubin | 0.0-1.0 | mg/dL |
| LDH | 100-220 | U/L |
| SGOT | 12-29 | U/L |
| SGPT | 9-41 | U/L |
| GGT | 11-55 | U/L |

TABLE IV
Baseline-Lipid/Cardiac Risk

| TEST | NORMAL RANGE | | UNITS |
|---|---|---|---|
| Cholesterol | 140-200 | | mg/dL |
| Triglycerides | 40-160 | | mg/dL |
| HDL-Cholesterol | see chart | | mg/dL |
| LDL-Cholesterol (Calc) | see chart | | mg/dL |
| Ferritin | see chart | | ng/dL |
| Apolipoprotein A1 | M: 115-190 | F: 115-220 | mg/dL |
| (APO A1) | | | |
| Apolipoprotein B | M: 70-160 | F: 60-150 | mg/dL |
| (APO B) | | | |

| | | |
|---|---|---|
| APOB/APO A1 Ratio | <1.0 | |
| Lipoprotein-a | 15-30 | mg/dL |
| Homocysteine | 4.0-15.0 | μmole/L |
| Fibrinogen | 200-400 | mg/dL |
| CPK | 41-186 | u/L |

TABLE V
BASELINE-Renal Profile

| Creatinine Clearance | | |
|---|---|---|
| Specimen Date: | | |
| Patient: | | |
| Patient | Measurement | Units |
| Patient Height | | Inches |
| Patient Weight | | Pounds |
| Patient Surface Area | | Square meters |
| Specimen Collection Time | | Hours |
| Urine Volume | | Millilters |
| Plasma Creatinine | | mg/dL |
| Urine Creatine | | mg/dL |

| Corrected Creatinine Clearance | | mL/min |
|---|---|---|
| TEST | NORMAL RANGE | UNITS |
| μ2-Microglobulin | 0.85-1.62 | mg/L |

TABLE VI
Post Tx (Treatment) 1

| | NORMAL RANGE | |
|---|---|---|
| TEST | (Invention score) | UNITS |
| Platelet Aggregation | (Score 3 or less) | |
| ADP | (Score 3 or less) | |
| EPI | (Score 3 or less) | |
| COLL | (Score 3 or less) | |
| THROMBIN | (Score 3 or less) | |
| PT | 11-15 | sec. |
| APTT | 16-25 | sec. |
| Magnesium | 1.2-2.3 | mEq/L |
| Calcium | 8.5-10.1 | mg/dL |
| Magnesium (ION) | 1.5-2.3 | mg/dL |
| Calcium (ION) | 3.9-5.5 | mg/dL |

Index of Abbreviations:

WBC=white blood cell

RBC=red blood cell

HgB=hemoglobin B

HCT=hematocrit

MCV=mean corpuscular volume

MCHC=mean corpuscular hemoglobin concentration

RDW=red cell distribution width

PLT-platelets

MPV=mean platelet volume

Lymph=lymphocytes

Mono=monocytes

Baso=basophylls

Eosin=eosinophylls

Segs=segmented neutrophylls

FL=femtoliter

SGOT(ALT)=Serum glutamate oxaloacetate transaminase Alanine Aminotransferase

SGOT (AST)=Serum glutamate oxaloacetate transamninase Aspartate Aminotransferase

BUN=blood urea nitrogen

LDH=lactate dehydrogenase

GGT=gamma glutamyltransferase

CPK=Creatine Kinase

M=10⁶ per ml³

K=10³ per ml³

TABLE VIIIA

| | |
|---|---|
| HDL | LDL |
| CHOLESTEROL (mg/dL) | CHOLESTEROL (mg/dL) |

| AGE) | | | AGE | | |
|---|---|---|---|---|---|
| (YRS) | MALE | FEMALE | (YRS) | MALES | FEMALES |
| 0-14 | 30-65 | 30-65 | 0-19 | 60-140 | 60-150 |
| 15-19 | 30-65 | 30-70 | 20-29 | 60-175 | 60-160 |
| 20-29 | 30-70 | 30-75 | 30-39 | 80-190 | 70-170 |
| 30-39 | 30-70 | 30-80 | 40-49 | 90-205 | 80-190 |
| over 40 | 30-70 | 30-85 | 50-59 | 90-205 | 90-220 |
| | | | 60-69 | 90-215 | 100-235 |
| | | | over 70 | 90-190 | 95-215 |

Values for African-Americans about 10 mg/dL higher

TABLE VIIIB
FERRITIN

| | | | | |
|---|---|---|---|---|
| MALES | 18-45 years | 22-340 ng/dL | >45 years | 22-415 ng/dL |
| FEMALES | 18-45 years | 6-115 ng/dL | >45 years | 15-200 ng/dL |

We claim:

1. A combination for amelioration of vascular insufficiency, comprising:
In a pharmaceutically acceptable carrier, a therapeutic dose of cystine and EDTA.

2. The claim according to claim 1, further comprising:
A therapeutic dose of Selenium.

3. The claim according to claim 2, further comprising:
A therapeutic dose of Vitamin C.

4. The claim according to claim 3, further comprising:
A therapeutic dose of Vitamin E.

5. The claim according to claim 4, further comprising:
A therapeutic dose of zinc.

6. A method of treatment of vascular insufficiency, comprising:
In a pharmaceutically acceptable carrier, administering cystine and EDTA.

7. The method according to claim 6, further comprising the following step:
Administering a therapeutic dose of Selenium.

8. The method according to claim 7, further comprising the following step:
Administering a therapeutic dose of Vitamin C.

9. The method according to claim 8, further comprising the following step:
Administering a therapeutic dose of Vitamin E.

10. The method according to claim 9, further comprising the following step:
Administering a therapeutic dose of zinc.

11. A method of measurement of efficacy and of treatment of vascular insufficiency, comprising:
Measuring glutathione levels in a patient, and upon determination of inadequate glutathione, administration of cystine;
Determining propensity to aggregation using the following steps:
Stabilizing a patient blood sample to prevent natural clotting;
Centrifuging said blood sample to generate a platelet fraction and extracting said platelet fraction;
Testing subparts of said platelet fraction with at least reagents selected from the group of ADP, epinephrine, collagen, and thrombin, and with saline as a control by combining said at least one reagent and said saline with said subpart of said platelet fraction in a cuvette comparable in size to a major artery;
Generating output from agitation and testing in a platelet aggregometer into which said at least two cuvettes have been placed;
Inspecting said cuvettes after agitation and testing to assure competent test results;
Rating each of said cuvettes for propensity to aggregation on a scale from 1 to 5, as set forth in Table I;
And upon determination of excess propensity to aggregation, administration of a therapeutic dose of EDTA and cystine, and intermittent continuation of said administration at a set first interval with repetition at a greater interval than said first interval of said determination step, until achievement of normal range of aggregation as set forth in Tables VI.

12. The method according to claim 11, further comprising the following step:
Monitoring of the achievement of normal range to ultimately restore glutathione levels to normal level, which should be approximately 200-400 micromoles/liter for plasma and red blood cells.

13. The method according to claim 11, further comprising the following step:
Administering a therapeutic dose of selenium.

14. The method according to claim 13, further comprising the following step:
Administering a therapeutic dose of Vitamin C.

15. The method according to claim 14, further comprising the following step:
Administering a therapeutic dose of Vitamin E.

16. The method according to claim 15, further comprising the following step:
Administering a therapeutic dose of zinc.

17. The method according to claim 14, further comprising the following step:
Monitoring creatinine excretion.

18. A method of monitoring the response to administration of EDTA for measurement of efficacy and treatment of vascular insufficiency, comprising:
Centrifuging said blood sample to generate a platelet fraction and extracting said platelet fraction;
Testing subparts of said platelet fraction with at least reagents selected from the group of ADP, epinephrine, collagen, and thrombin, and with saline as a control by combining said at least one reagent and said saline with said subpart of said platelet fraction in a cuvette comparable in size to a major artery;
Generating output from agitation and testing in a platelet aggregometer into which said at least two cuvettes have been placed;
Inspecting said cuvettes after agitation and testing to assure competent test results;
Rating each of said cuvettes for propensity to aggregation on a scale from 1 to 5, as set forth in Table I;
And upon determination of excess propensity to aggregation, administration of a therapeutic dose of EDTA and cystine, and intermittent continuation of said administration at a set first interval with repetition at a greater interval than said first interval of said determination step, until achievement of normal range of aggregation as set forth in Tables VI.

19. The method according to claim 18, further comprising the following step:
Measuring glutathione levels in a patient, and upon determination of inadequate glutathione, administration of cystine.

20. The method according to claim 19, further comprising the following step:
Monitoring of the achievement of normal range to ultimately restore glutathione levels to normal level, which should be approximately 200-400 micromoles/liter for plasma and red blood cells.

21. The method according to claim 18, further comprising the following step:
Administering a therapeutic dose of selenium.

22. The method according to claim 21, further comprising the following step:
Administering a therapeutic dose of Vitamin C.

23. The method according to claim 22, further comprising the following step:
Administering a therapeutic dose of Vitamin E.

24. The method according to claim 23, further comprising the following step:
Administering a therapeutic dose of zinc.

25. The method according to claim 24, further comprising the following step:
Monitoring creatinine excretion.

26. A method of measurement of efficacy and of treatment of vascular insufficiency, comprising:
Measuring glutathione levels in a patient, and upon determination of inadequate glutathione, administration of cystine;
Monitoring of the achievement of normal range to ultimately restore glutathione levels to normal level, which should be approximately 200-400 micromoles/liter for plasma and red blood cells;
Determining propensity to aggregation using the following steps:
Stabilizing a patient blood sample to prevent natural clotting;
Centrifuging said blood sample to generate a platelet fraction and extracting said platelet fraction;
Testing subparts of said platelet fraction with at least reagents selected from the group of ADP, epinephrine, collagen, and thrombin, and with saline as a control by combining said at least one reagent and said saline with said subpart of said platelet fraction in a cuvette comparable in size to a major artery;
Generating output from agitation and testing in a platelet aggregometer into which said at least two cuvettes have been placed;
Inspecting said cuvettes after agitation and testing to assure competent test results;
Rating each of said cuvettes for propensity to aggregation on a scale from 1 to 5, as set forth in Table I;
And upon determination of excess propensity to aggregation, administration of a therapeutic dose of EDTA and cystine, and intermittent continuation of said administration at a set first interval with repetition at a greater interval than said first interval of said determination step, until achievement of normal range of aggregation as set forth in Tables VI;
Measuring total serum calcium, ionized calcium, total magnesium, and ionized magnesium; and
Monitoring creatinine excretion.

27. The method according to claim 26, further comprising the following step:
Administering a therapeutic dose of selenium.

28. The method according to claim 27, further comprising the following step:
Administering a therapeutic dose of Vitamin C.

29. The method according to claim 28, further comprising the following step:
Administering a therapeutic dose of Vitamin E.

30. The method according to claim 29, further comprising the following step:
Administering a therapeutic dose of zinc.
